(19) United States
(12) Patent Application Publication
(10) Pub. No.: US 2023/0201618 A1
(43) Pub. Date: Jun. 29, 2023

(54) MAGNETO-ENDOSOMALYTIC THERAPY FOR CANCER

(71) Applicant: UNIVERSITY OF WASHINGTON, Seattle, WA (US)

(72) Inventor: Xiaohu Gao, Seattle, WA (US)

(73) Assignee: UNIVERSITY OF WASHINGTON, Seattle, WA (US)

(21) Appl. No.: 17/998,798

(22) PCT Filed: May 21, 2021
(86) PCT No.: PCT/US2021/033623

§ 371 Date: Nov. 14, 2022

Related U.S. Application Data
(60) Provisional application No. 63/030,072, filed on May 26, 2020.

(51) Int. Cl. A61N 2/00; A61N 2/06; A61K 41/00; A61K 47/54

(52) U.S. Cl. CPC A61N 2/004; A61N 2/06; A61K 41/0028; A61K 47/542

(57) ABSTRACT

Methods for inducing cell death by magneto-endosomalytic therapy using magnetic nano articles. The methods include inducing cell death, inducing cell death in a subject, and application of the methods to killing tumor cells in a subject.

### CROSS-REFERENCE TO RELATED APPLICATION

This application claims the benefit of U.S. Application No. 63/030,072, filed May 26, 2020, expressly incorporated herein by reference in its entirety.

### BACKGROUND OF THE INVENTION

The surge of nanotechnology research in the past 20 years has produced tremendous insights in the design principles (e.g., optimal NP size, shape, surface charge, and targeting ligand) for efficient molecular imaging and therapy. Despite the in-depth understanding of these advanced technologies, their clinical translation has been largely negligible. Only a handful of particle types such as albumin aggregates, liposomes, biodegradable polymers, and iron oxides are currently used in humans, and they were largely developed long before the nanotechnology era. For NP-based therapeutics, clinical translation is particularly challenging due to many reasons, but most prominently, the structural and chemical complexity of nanoformulations, inability to address drug side effects (even with active targeting), and potential nanotoxicity. As a result, none of the signature nanostructures reported in recent years such as Au nanostructures, semiconductor quantum dots, and highly monodisperse iron oxides is approved for patient care. Only the conventional iron-dextran complexes and polydisperse iron oxides developed decades ago are currently in clinical use as an iron-deficiency supplement and magnetic resonance imaging (MRI) contrast agent, respectively.

Despite the advances in the use of magnetic nanoparticles above, a need exists for improved therapeutic methods for their use. The present invention seeks to fulfill this need and provides further related advantages.

### SUMMARY OF THE INVENTION

The present invention provides methods for inducing cell death by magneto-endosomalytic therapy using magnetic nanoparticles.

In one embodiment, the invention provides a method for inducing cell death, comprising:

(a) contacting a cell with a magnetic nanoparticle;

(b) after a pre-determined period of time, subjecting the cell to a parallel magnetic field for a time sufficient to achieve cell death.

In another embodiment, the invention provides a method for inducing cell death in a subject, comprising:

(a) administering an effective amount of magnetic nanoparticles to a subject;

(b) after a pre-determined period of time, subjecting the subject to a localized parallel magnetic field for a time sufficient to achieve cell death.

In another embodiment, the invention provides a method for killing tumor cells in a subject, comprising;

(a) administering an effective amount of magnetic nanoparticles to a subject;

(b) after a pre-determined period of time, subjecting the solid tumor to a localized parallel magnetic field for a time sufficient to achieve cell death and decrease the size of the tumor.

In certain embodiments, the magnetic nanoparticle is an iron oxide nanoparticle, such as dextran- or polyethylene glycol-coated magnetic nanoparticle. In certain embodiments, the magnetic nanoparticle is a magnetic resonance imaging agent, such as a clinically-approved magnetic nanoparticle.

In certain embodiments, the magnetic nanoparticle further includes a targeting agent for selective cell targeting.

In the practice of the methods of the invention, the magnetic nanoparticles are administered by local injection, subcutaneous injection, and intravenous injection. In certain embodiments, the magnetic nanoparticles are administered by injection into the solid tumor.

In the practice of the methods of the invention, the parallel magnetic field is provided by a first and a second permanent magnet. In these embodiments, the cells to be treated are positioned within parallel magnetic field provided by the first and second permanent magnets (e.g., the parallel magnetic field provided by the first and second permanent magnets is positioned on opposite sides of a solid tumor).

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1A is a schematic representation of the method of the invention. MNPs are taken up by cancer cells, a process that has been routinely demonstrated with or without a targeting ligand. The iron oxide-based MNPs are biocompatible. However, sandwiching the cells with endocytosed MNPs between two simple magnets leads to MNP self-assembly that ruptures endosomes, triggering cell death. The dotted parallel lines with arrowheads in the center indicate magnetic field lines.

FIG. 1B compares cell viability for cervical cancer cells, HeLa, in accordance with a representative method of the invention. Cell viability was assessed (calcein-AM staining of live cells; ethidium homodimer-1 staining of dead cells) after treating cells with 2 parallel magnets alone, MNP alone, MNP with one magnet, and MNP with two parallel magnets. Only the combination of MNP with two magnets showed dose-dependent cell death (bottom right panel, measurements n=6, from different wells). At 500 μg mL⁻¹ (iron concentration during MNP incubation), the vast majority of the cells were killed.

FIG. 2A is an image tracking MNPs and lysosomes inside HeLa cells treated by the method of the invention. Endosomes/lysosomes in cells treated by the method disappeared, whereas MNPs alone and the combination of MNPs and a single magnet left these intracellular compartments intact.

FIG. 2B is an image illustrating intracellular pH measured by a pH-sensitive dye (pHrodo™ Green AM). Endosome/lysosome rupture led to a pH drop inside cells.

FIGS. 3A-3D illustrate treating tumor organoids by a representative method of the invention. FIG. 3A illustrates representative fluorescence images and

FIG. 3B compares quantitative fluorescence intensity measurements of tumor organoids incubated with fluorescently labeled MNPs. The MNPs with a targeting ligand, DUPA, showed significantly enhanced uptake (n=3, ****P<0.0001). FIG. 3C compares the size of tumor organoids untreated, treated with the magnetic field (MF) alone, MNP alone, MNP-DUPA alone, MELT with non-targeted MNPs, or MELT with DUPA-targeted MNPs. The two black arrows indicate the days when tumor organoids were placed in the parallel magnetic fields for 1.5 h. The representative method using both non-targeted MNPs and DUPA-targeted MNPs showed treatment efficacy. Compared to the untreated group whose size increased by 69.5%, the growth of the organoids treated with non-targeted MNPs in the presence of the magnetic fields slowed down (size increase by 27.1%), whereas the size of the organoids treated with DUPA-targeted MNPs in the presence of the magnetic fields reduced by 27.8%. The other treatment groups showed no-effects virtually. Measurements n=8 from different samples, ***P<0.001; ****P<0.0001. FIG. 3D compares quantitative cell viability measurements of the tumor organoids 7 days after the treatments described in FIG. 3C (n=8 from distinct samples, ****P<0.0001, #### ****P<0.0001).

FIG. 4 presents images demonstrating MNP self-assembly in parallel magnetic fields: assembly of fluorescently (Cy3) labeled Feraheme® MNPs without a magnetic field (MF), in the presence of one magnet, or two parallel magnets. When sandwiched between two magnets, MNPs self-assemble into micrometer sized rods or aggregates (see inset).

FIG. 5 illustrates treating MDA-MB-231 breast cancer cells by a representative method of the invention. Similar to FIG. 1B, cell viability was assessed (calcein-AM staining of live cells; ethidium homodimer-1 staining of dead cells) after treating cells with the parallel magnetic field (MF) alone, MNPs alone, MNPs with one magnet, and MNPs with two parallel magnets. Only the combination of MNPs with two magnets showed dose dependent cell death (bottom right panel, measurements n=6 from distinct samples). At 500 μg mL⁻¹ (iron concentration during MNP incubation), the vast majority of the cells were killed.

FIG. 6 illustrates treating LNCAP prostate cancer cells by a representative method of the invention. Similar to FIGS. 1B and 5, cell viability was assessed (calcein-AM staining of live cells; ethidium homodimer-1 staining of dead cells) after treating cells with the parallel magnetic field (MF) alone, MNPs alone, MNPs with one magnet, and MNPs with two parallel magnets. Only the combination of MNPs with two magnets showed dose dependent cell death (bottom right panel, measurements n=6 from distinct samples). At 500 μg mL⁻¹ (iron concentration during MNP incubation), the vast majority of the cells were killed.

FIG. 7 illustrates treating 22RV1 prostate cancer cells by a representative method of the invention. Similar to FIGS. 1B, 5, and 6, cell viability was assessed (calcein-AM staining of live cells; ethidium homodimer-1 staining of dead cells) after treating cells with the parallel magnetic field (MF) alone, MNPs alone, MNPs with one magnet, and MNPs with two parallel magnets. Only the combination of MNPs with two magnets showed dose dependent cell death (bottom right panel, measurements n=6 from distinct samples). At 500 μg mL⁻¹ (iron concentration during MNP incubation), the vast majority of the cells were killed.

### DETAILED DESCRIPTION OF THE INVENTION

The present invention provides methods for inducing cell death by magneto-endosomalytic therapy using magnetic nanoparticles. The methods include inducing cell death, inducing cell death in a subject, and application of the methods to killing tumor cells in a subject.

In one embodiment, the invention provides a method for inducing cell death, comprising:

(a) contacting a cell with a magnetic nanoparticle;

(b) after a pre-determined period of time, subjecting the cell to a parallel magnetic field for a time sufficient to achieve cell death.

In another embodiment, the invention provides a method for inducing cell death in a subject, comprising:

(a) administering an effective amount of magnetic nanoparticles to a subject;

(b) after a pre-determined period of time, subjecting the subject to a localized parallel magnetic field for a time sufficient to achieve cell death.

In a further embodiment, the invention provides a method for killing tumor cells in a subject, comprising;

(a) administering an effective amount of magnetic nanoparticles to a subject;

(b) after a pre-determined period of time, subjecting the solid tumor to a localized parallel magnetic field for a time sufficient to achieve cell death and decrease the size of the tumor.

As used herein the term “pre-determined period of time” refers to period of time, after cell contact or administration to a subject, sufficient to allow the magnetic nanoparticle to accumulate in the cell or cells of desired tissue to be treated. The pre-determined period of time will depend on the cell or cells of desired tissue and the mode of contact or administration. The pre-determined period of time is readily determined by the skilled person.

As used herein, the term “parallel magnetic field” refers to a magnetic field generated by a pair of permanent magnets positioned statically (no movement) or dynamically (movement about the targeted cells, e.g., rotation) or an electromagnet(s) (magnetic field generated from an electric current, optionally pulsed at low frequency, e.g., less than about 100 Hz). It will be appreciated that the parallel magnetic field can be generated from an electric field. Electric fields are created by electric charges, or by time-varying magnetic fields. Electric fields and magnetic fields are both manifestations of electromagnetic force.

In the methods of the invention, the nature of the magnetic nanoparticle is not critical so long as the magnetic nanoparticle that can be taken up into a cell by, for example, endocytosis. Representative magnetic nanoparticles have a size from about 1 to about 1000 nm.

In certain embodiments, the magnetic nanoparticle is an iron oxide nanoparticle. Other suitable nanoparticles include metals such as cobalt, nickel and their alloys.

In certain embodiments, the magnetic nanoparticle is a dextran- or polyethylene glycol-coated magnetic nanoparticle. Other suitable nanoparticles include polymer-coated particles, inorganic-coated (C and Si) particles, and polymer encapsulated magnetic nanoparticle clusters (i.e., a single polymer-coated particle that includes multiple MNPs encapsulated therein).

In certain embodiments, the magnetic nanoparticle is a magnetic resonance imaging agent.

In certain embodiments, the magnetic nanoparticle is a clinically approved magnetic nanoparticle. In certain of these embodiments, the magnetic nanoparticle is Feraheme® (Feraheme® is a non-stoichiometric magnetite (superparamagnetic iron oxide) coated with polyglucose sorbitol carboxymethylether. Feraheme® injectable solution (ferumoxyltol injection) is a sterile aqueous colloidal product formulated with mannitol for intravenous administration as a magnetic resonance imaging contrast media.

The magnetic nanoparticles useful in the method are taken up by cells of interest by, for example, endocytosis. Selectivity in magnetic nanoparticle uptake by the cells may be passive or by active targeting. It is well known that cancer cells, such as in tumors, grow more rapidly than normal cells. Rapid cell growth facilitates a selective uptake of the magnetic nanoparticles in rapidly proliferating cancer cells. It is also well known that cancer cells are more permeable and have vascularity that promote selective uptake relative to normal cells. Alternatively, the magnetic nanoparticles can be directed to cells of interest through active targeting. In certain embodiments, the magnetic nanoparticle useful in the methods further comprises a targeting agent for selective cell targeting. Suitable targeting agents include ligands that target specific cell surface receptors. Such targeting agents include small molecules and peptides, as well as antibodies and their functional fragments, and aptamers, each of which can be associated with the magnetic nanoparticle by covalent or other associative techniques.

Selectivity of the methods of the invention can also be achieved by localization of the magnetic field (e.g., the positioning of the two magnets) such that the magnetic field is localized only at the site designated for cell death to the exclusion of other sites not designated for treatment that may include cells that have taken up magnetic nanoparticles. As described herein, shielding may be used to protect sites not designated for treatment that may include cells that have taken up magnetic nanoparticles.

In the practice of the methods of the invention, the magnetic nanoparticles may be administered by local injection (e.g., image-guided injection), subcutaneous injection, and intravenous injection. In certain embodiments, the magnetic nanoparticles are administered by injection into the solid tumor (intra-tumor targeted).

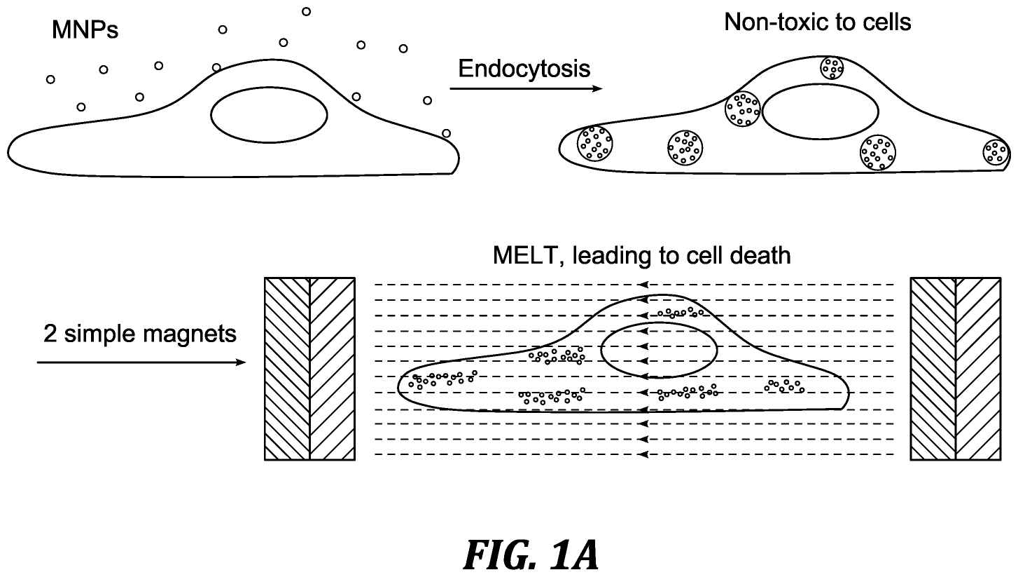

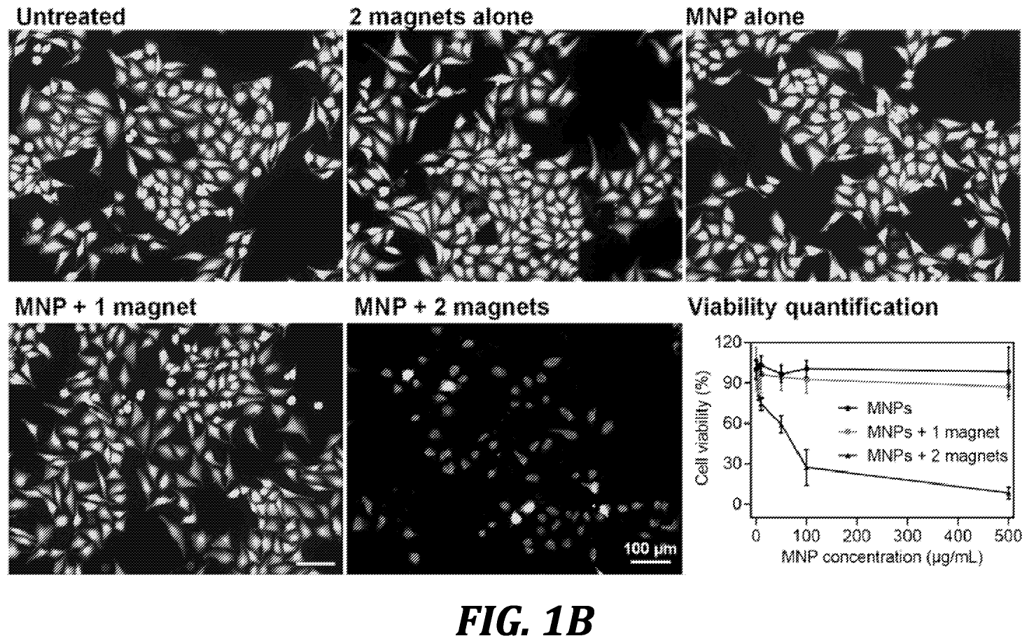

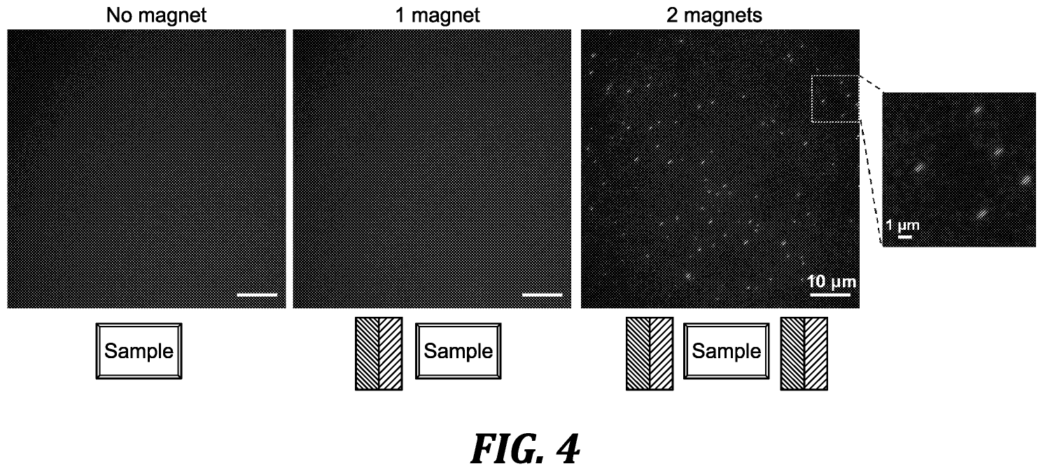

In the practice of the methods of the invention, the parallel magnetic field is provided by a first and a second permanent magnet. See, for example, FIGS. 1A and 4. Other suitable magnets include electromagnets. In certain embodiments, the magnetic field is generated by an electric field. In certain embodiments, the parallel magnetic field has a field strength up to about 3 T (e.g., 0.25 T).

In the methods of the invention, the cells to be treated are positioned within parallel magnetic field provided by a first and a second permanent magnet (or an electromagnet, or magnetic field generated by an electric field). See, for example, FIGS. 1A and 4

In certain embodiments, the parallel magnetic field provided by a first and a second permanent magnet is positioned on opposite sides of the solid tumor. In certain of these embodiments, the subject's liver and optionally spleen, are shielded from the magnetic field.

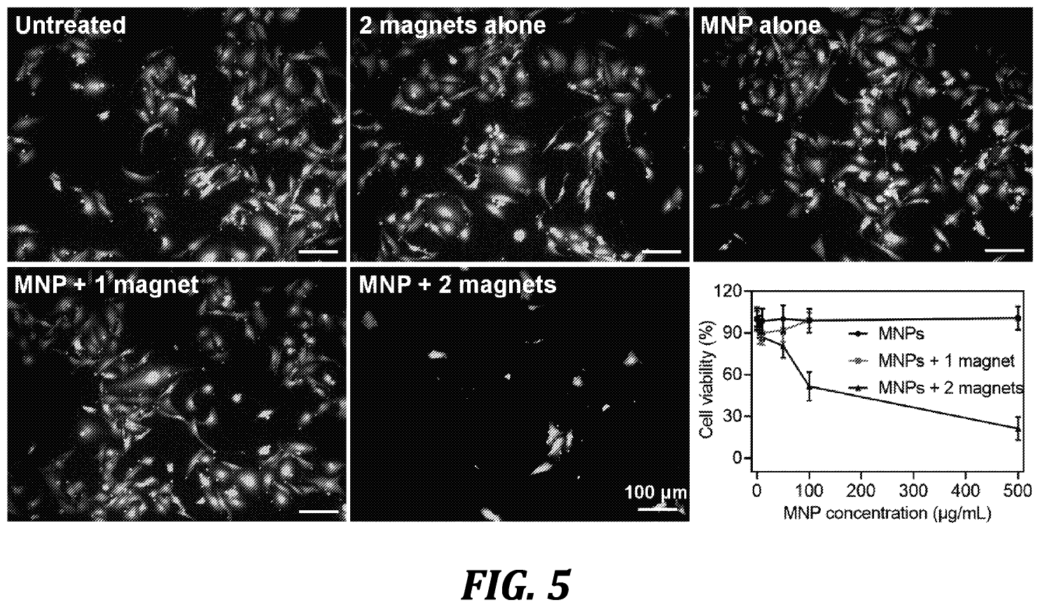

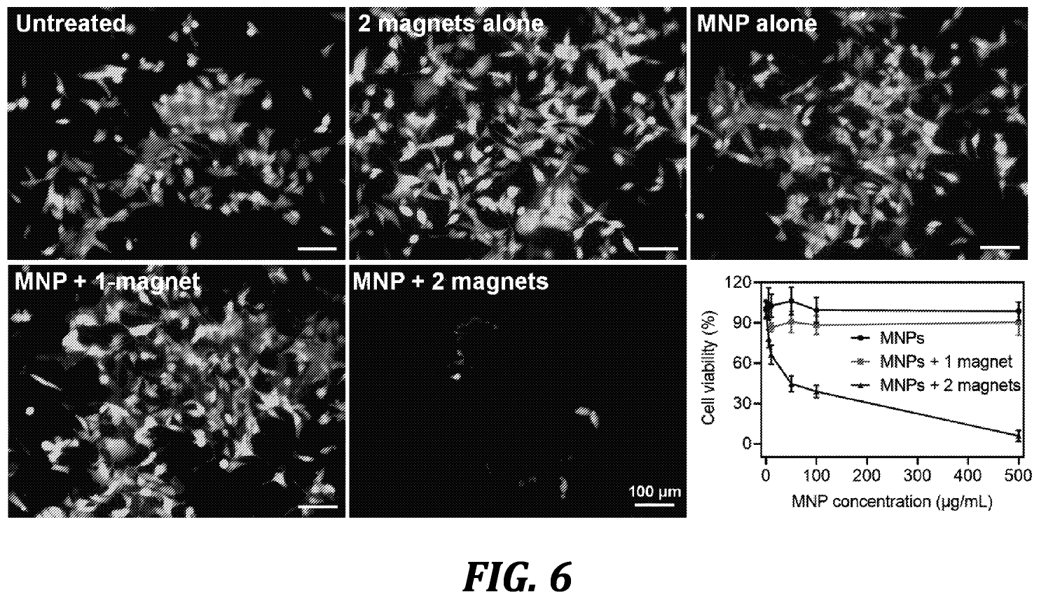

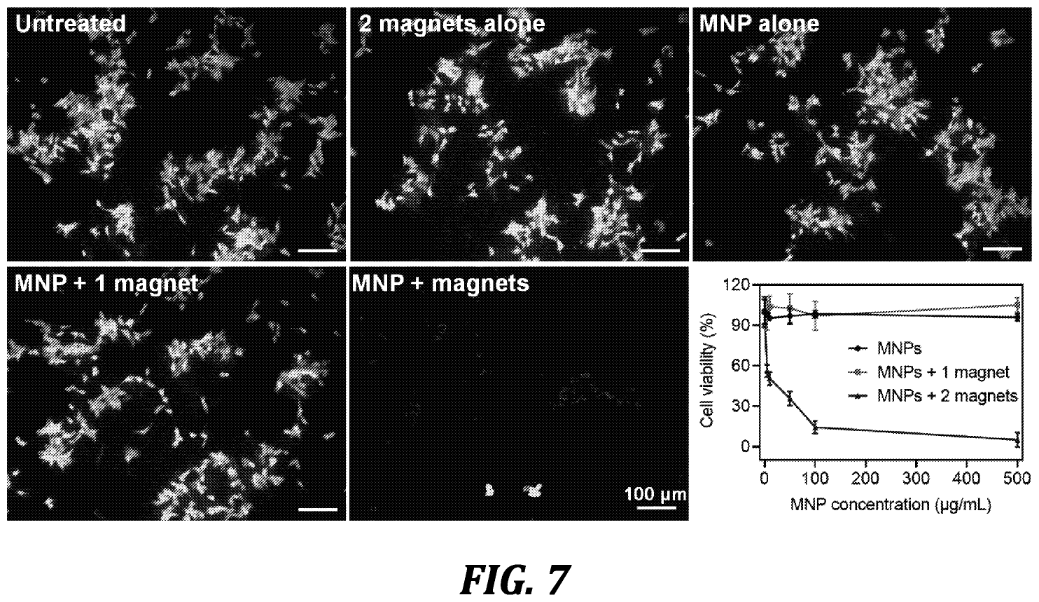

The nature of the cells useful in the methods of the invention are not limited so long as the magnetic nanoparticle can be taken up into the cell. In certain embodiments, the cells are cancer cells (e.g., human prostate (LNCaP, 22RV1, PC-3, and DU-145), cervical (HeLa), breast (MDA-MB-231), glioblastoma (U87), and colorectal (HCT) cancer cells). Results for cell killing for MDA-MB-231 breast cancer cells, LNCAP prostate cancer cells, and 22RV1 prostate cancer cells are shown in FIGS. 5-7, respectively.

In certain embodiments, the cells are the cells of a cancerous solid tumor.

In the practice of the methods of the invention, the parallel magnetic field induces intracellular self-assembly of the magnetic nanoparticles resulting in cell death. In certain embodiments, cell death is achieved by lysosomal membrane permeabilization. In other embodiments, cell death is achieved by lysosomal-dependent cell death (LDCD) (e.g., apoptosis, autophagic cell death, necrosis).

The methods of the invention do not include introduction of one or more traditional therapeutic agents (e.g., small molecule chemotherapeutic or biological agent, for example, a polypeptide, protein, protein fragment, or nucleic acid, such as a DNA or RNA) into the cells to be treated. The methods of the invention rely solely on the introduction of the magnetic nanoparticles to facilitate therapeutic action (i.e., cell death), optionally in combination with a non-therapeutic targeting agent.

The following is a description of representative methods of the invention using magnetic nanoparticles to induce cell death.

The present invention provides a new mode of therapy for all cancer cell types, Magneto-EndosomaLytic Therapy for cancer (MELT cancer). Although the methods induce cell death using virtually all types of MNPs, the use of an FDA-approved MNP (Feraheme®) is described below. As shown in FIG. 1A, MNPs can be delivered to cells via endocytosis with or without targeting ligands, which have been routinely demonstrated for iron oxide NPs (and many others). The safety of dextran- and PEG-coated iron oxide NPs is well established, with a few types having received regulatory approvals. Surprisingly, sandwiching the cells between two simple permanent magnets that create a parallel magnetic field turns the non-toxic imaging agent into a highly effective therapeutic compound. The MNPs self-assembled into rod-like structures that trigger cell death regardless of cell type (see FIG. 4). In contrast to magnetothermal therapy, the methods of the invention do not require high-power high-frequency energy inputs to achieve cell death. Furthermore, the magnets used in the methods can be selectively placed on both sides of the targeted disease sites while leaving healthy organs (e.g., liver that often takes up the vast majority of administered drugs) intact. The method can be readily carried out and positive clinical outcomes achieved for patients during bed rest.

To evaluate the effectiveness and generalizability of MELT in treating cancer, Feraheme® MNPs were added to multiple types of cancer cell lines such as human prostate (LNCaP and 22RV1), cervical (HeLa), and breast (MDA-MB-231) cancer cell lines. After cell uptake, the cell culture dishes were placed between two permanent magnets as schematically illustrated in FIG. 1A. Remarkably, dose-dependent apoptosis was observed across all cell types tested including the triple-negative breast cancer cell, MDA-MB-231 (see FIGS. 1B and 5-7). In contrast, the application of the Feraheme® MNPs or dual magnets alone did not produce detectable toxicity under the current experiment condition (2.38 pg of Fe³⁺ per cell, and 0.25 T magnetic field), as expected, because these conditions are well in the range of typical MRI studies. Interestingly, combining Feraheme® MNPs of the same concentration with a single magnet, the common condition used for cell isolation, did not produce significant cell death either. The magnetic pulling force was too weak to create mechanical damage to cells. Recent studies have estimated a strong force of approximately 500 pN would be needed to damage cells, which requires a magnetic field gradient of 1,000 s T m⁻¹ (O. Lunov, M. Uzhytchak, B. Smolková, M. Lunova, M. Jirsa, N. M. Dempsey, A. L. Dias, M. Bonfim, M. Hof, P. Jurkiewicz, Y. Petrenko, S̆. Kubinová, A. Dejneka, Cancers 2019, 11, 1873). The method of the invention with the simple change of adding a second magnet to create a homogeneous magnetic field (FIG. 1A) and a negligible field gradient, however, turned the biocompatible MNPs to a lethal compound for cancer cells.

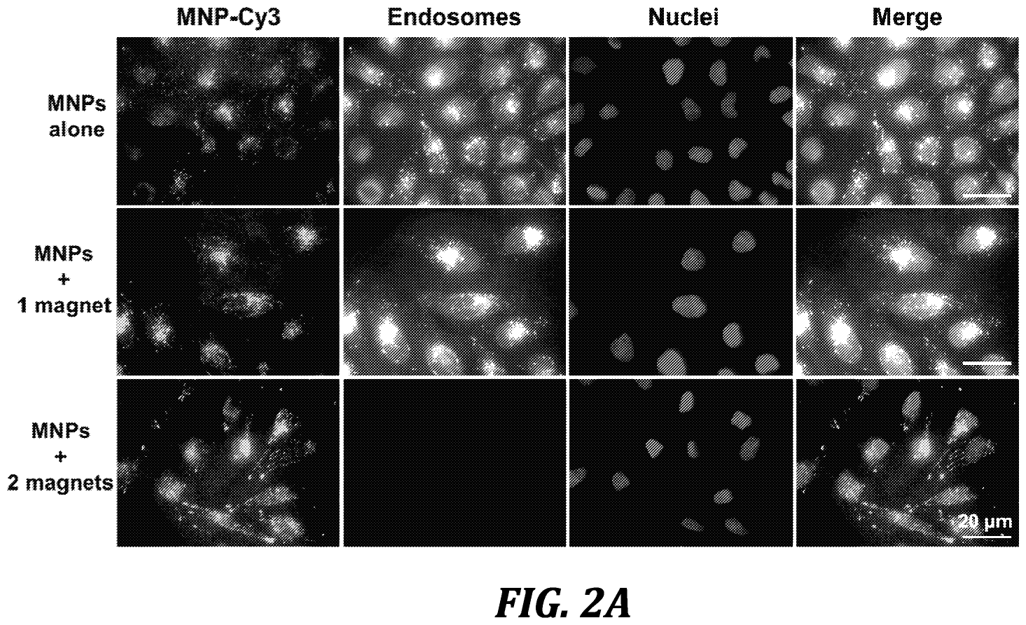

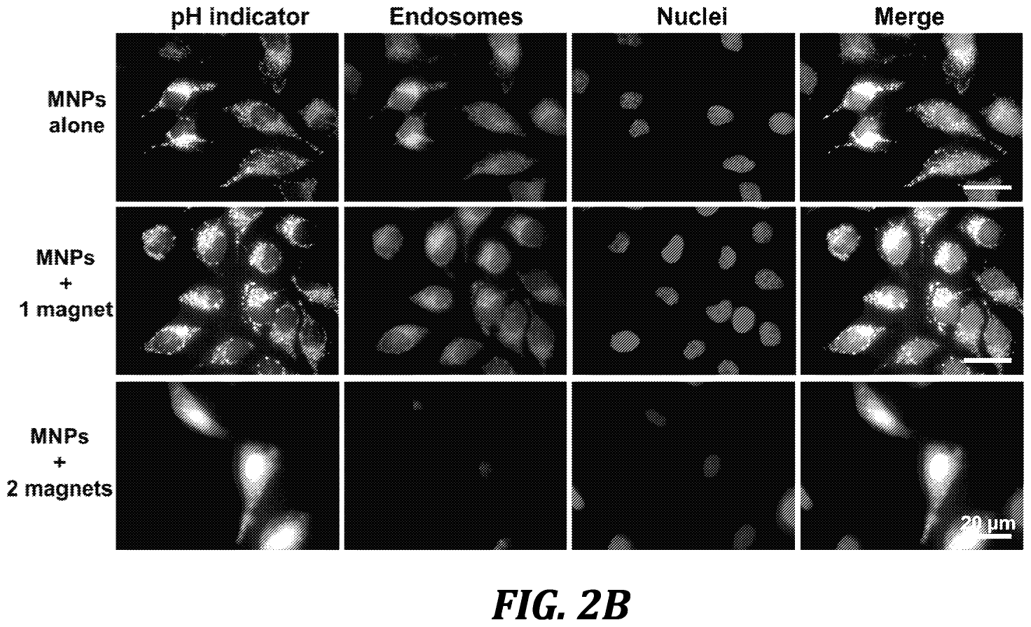

The mode of action of method of the invention was investigated using fluorescence microscopy. In response to external magnetic fields, MNPs have been previously shown to exert a force to disrupt the cell plasma membrane, cytoskeleton, and endosomes. These studies, however, require either large NPs and extremely high field strength to achieve the desired mechanical forces, or pulsed or alternating magnetic fields that are complicated to construct, limiting the potential for downstream clinical translation. In contrast, the method of the invention works for virtually all types of MNP and only requires two low-cost permanent magnets. To visualize the cell entry and intracellular location of MNPs in the method, Feraheme® was fluorescently labeled with Cy3 (red) and incubated with cells. Once taken inside the cells, the MNPs showed a punctate intracellular distribution (FIG. 2A), a signature of endocytosis, which was further confirmed by co-labeling cells with an Endotracker. Without a magnet or with a single magnet placed near the cells (like the condition used for magnetic cell separation), co-localization of the MNPs and Endotracker was observed throughout the entire experiment. Strikingly, sandwiching the cells between two magnets led to the complete disappearance of endosomes, demonstrating the endosomalytic power of the method.

The role of lysosome in programmed cell death has been well established. In fact, after its discovery in 1955, lysosome was even proposed to be the “suicide bag.” Lysosomal membrane permeabilization (LMP) results in translocation of lysosomal compounds such as protons and cathepsins into the cytoplasm. These compounds can induce lysosomal-dependent cell death (LDCD) through multiple pathways including apoptosis, autophagic cell death, and necrosis, depending on the degree of LMP. In response to the lysosome disappearance shown in FIG. 2A, the intracellular pH was further measured using a pH sensing dye (pHrodo™ Green AM Intracellular pH Indicator). Compared to cells treated with MNPs alone, or MNP+magnet fields created by a single magnet, cells treated with MNPs and two magnets (MELT) showed reduced pH in the cytosol (FIG. 2B), confirming the damage of lysosomal membrane and the subsequent LDCD.

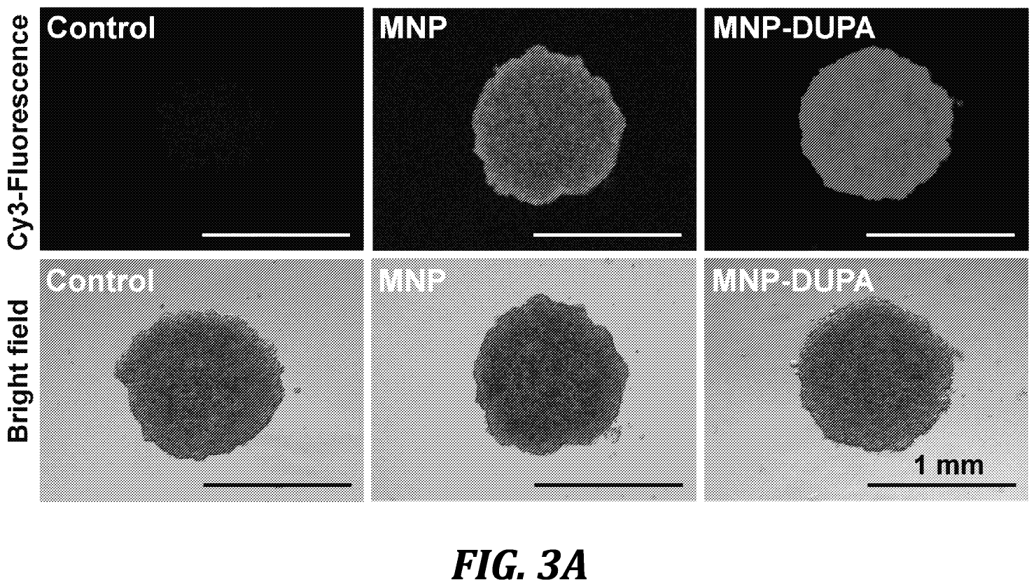

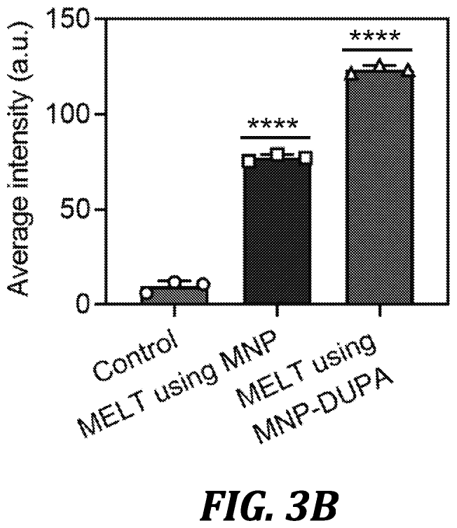

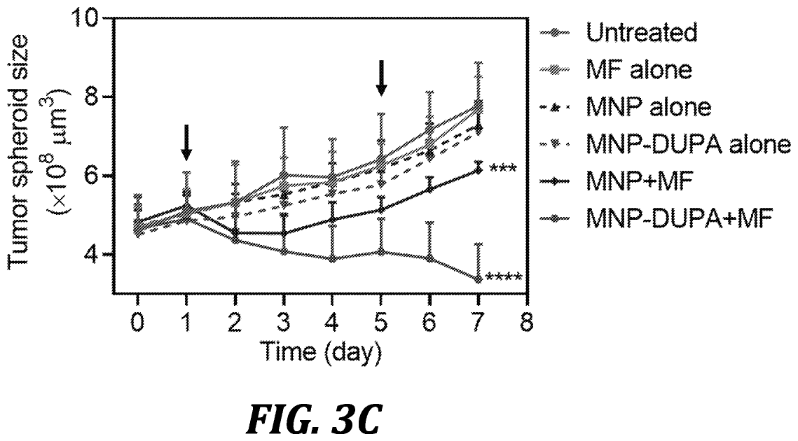

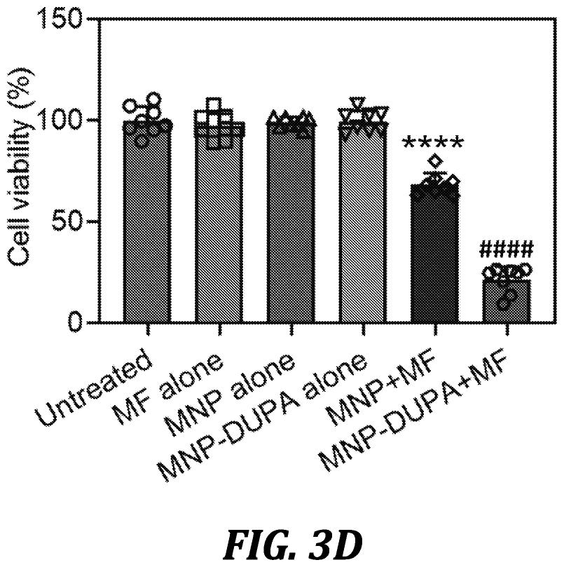

To evaluate the potential therapeutic effect of MELT in solid tumors, an LNCaP 3-D organoid model was used. Organoids are three-dimensional, self-organized, miniature structures that recapitulate the tissue architecture and maintain the genomic alterations of solid tumors in vivo. Recently, more and more evidence has shown that advanced patient-derived organoids (PDOs) can be used to accurately predict drug responses in a personalized treatment setting. As described herein, when the tumor organoids reached a size of approximately 1 mm in diameter, they were incubated with MNPs or MNPs conjugated to DUPA (2-[3-(1,3-dicarboxy propyl) ureido] pentanedioic acid), a small-molecule ligand against PSMA (prostate-specific membrane antigen). The DUPA ligand enhanced the cell uptake of Feraheme® MNPs by 60.2% (FIGS. 3A and 3B). The MNP-treated organoids were placed in the parallel magnets as described above (on day 1 and 5, each time for 1.5 h). Compared to the untreated, organoids treated with MNPs alone, magnetic field (MF) alone, or MNP-DUPA alone, whose volume increased by approximately 60-70% in a week, the non-targeted MNP-treated organoids in the presence of parallel MFs only increased by 27.1%, whereas the volume of organoids treated with DUPA-targeted MNPs combined with the MFs was reduced by 27.8% (FIG. 3C). The therapeutic effect was further confirmed with the cell viability assay, showing the reduction of viable cells by 31.6% for the non-targeted MNP treatment and by 78.7% for the DUPA-targeted MNP treatment compared to the untreated control (FIG. 3D).

The present invention provides a simple and effective mode of cancer treatment by combining biocompatible MNPs with two low-cost permanent magnets. Intracellular nanoparticle self-assembly directed by the static parallel magnetic fields results in cell death for both cultured cells and 3-D organoids. The method provides transformative features: (1) the method provides a new avenue to capitalize on the NP design principles (e.g., size, surface charges, targeting ligands) for clinical translation and (2) the method provides that precise targeting can be achieved on two levels, the molecular level based on targeting ligands and the body level based on the placement of the parallel magnets, as neither one by itself is sufficient to avoid the treatment side effects. For example, even with a selective targeting ligand, the vast majority of nanotherapeutics still accumulate in the liver and spleen (e.g., sandwiching body parts that may have tumor cells between magnets while avoiding the liver) substantially reduces the toxicity, not to mention more selective placement of the magnets. Indeed, exquisite shaping of magnetic fields can even move magnetic robots in vivo for eye surgery. As described herein, it is believed that the method of the invention may replace the majority of the current nanoparticle delivery systems for cytotoxic chemical drugs because the method provides the same tumor cell killing function while offering higher temporal and spatial precision.

As described herein, the method of the invention is demonstrated effective using cultured cells and tumor-mimicking 3D organoids. With the safety data well-established for both the MNPs and the magnetic fields, the method of the invention can facilitate more rapid clinical trials, such as image-guided local injection in tumors that are inaccessible to surgical scalpels, systemic administration coupled with the molecular and/or magnetic field targeting, and combination with other types of treatment especially immune responses triggered by localized tumor killing and inflammation.

The following materials and methods are provided to illustrate, not limit, the method of the invention.

### Materials and Methods

Materials. Feraheme®, also known as ferumoxytol injection for IV and manufactured by AMAG Pharmaceuticals, Inc., Waltham Mass., was received from the University of Washington, Department of Radiology. Feraheme® is a sterile aqueous colloid of superparamagnetic iron oxide surface coated with polyglucose sorbitol carboxymethylether for intravenous administration as an MRI contrast media. Neodymium magnets were purchased from K&J Magnetics Incorporated. Unless otherwise mentioned, all common chemicals were purchased from Sigma-Aldrich. Hoechst 33258, Lysotracker Green®, Lysotracker DeepRed®, pHrodo™ Green AM Intracellular pH Indicator, and LIVE/DEAD™ Viability/Cytotoxicity Kit were purchased from ThermoFisher Scientific. Cyanine3 (Cy3)-amine was bought from Lumiprobe Corporation. CellTiter 96® Aqueous One Solution Cell Proliferation Assay (MTS) was obtained from Promega Corporation.

Feraheme® labeling and DUPA conjugation. Feraheme® were chemically labeled with cyanine-3-amine (Cy3-amine) using the standard N-(3-dimethylaminopropyl)-N-ethylcarbodiimide/N-hydroxysuccinimide (EDC/NHS) chemistry. Briefly, Feraheme® (1 mg) was dissolved in a MES buffer, followed by addition of NHS (1 mg) and NHS (1.5 mg). After 15 min incubation, the MNPs were quickly purified on a desalting column and added to the Cy3-amine solution. The incubation was allowed to last for 2 h at room temperature followed by purification with a desalting column. Aminated DUPA (W. Tai, J. Li, E. Corey, X. Gao. A ribonucleoprotein octamer for targeted siRNA delivery. Nat. Biomed. Eng. 2018, 2, 326-337) was conjugated to Feraheme® surface using the same procedure.

Cell culture. Cell lines were purchased from American Type Culture Collection (ATCC). LNCaP human prostate cancer cells (ATCC# CRL-1740) and 22RV1 human prostate cancer cells (ATCC# CRL2505) were cultured in RPMI-1640 medium containing 10% fetal bovine serum (FBS), penicillin (100 units mL⁻¹), and streptomycin (100 μg mL⁻¹). HeLa human cervical cancer cells (ATCC# CCL-2) were cultured in Eagle's minimal essential medium supplemented with FBS and penicillin/streptomycin. MDA-MB-231 human triple-negative breast cancer cells (ATCC# HTB-26) were culture in L-15 medium containing FBS and penicillin/streptomycin. All the cells were grown in a cell incubator supplied with 5% CO₂, except that MDA-MB-231 cells were cultured in airtight flasks.

Quantification of Feraheme® cell uptake. Cells with endocytosed MNPs were counted using a hemocytometer, before concentrated hydrochloric acid and hydrogen peroxide were added to completely dissolve and oxidize the internalized MNPs to Fe³⁺. Finally, Fe³⁺ concentration in solution was measured based on UV/vis absorption at 298 nm compared to standards.

MELT cancer cells. The cytotoxicity of MNP in LNCaP cells, 22RV1 cells, HeLa cells and MDA-MB-231 cells was assessed using the Live/Dead cell staining. Briefly, cells were seeded in 24-well glass-bottom plates (20,000 cells per well). After overnight culturing, cells in different wells were divided into 5 experimental groups: untreated, treated with 2 magnets only, treated with MNP only, treated with MNP and 1 magnet, and treated with MNP and 2 magnets. In the 5 groups, cells were incubated with the medium with or without the MNPs (500 μg mL⁻¹ based on Fe) for 3 h. Fresh media were replaced, and the cells were placed in different magnetic fields (1 magnet or 2 magnets) for 1.5 h. After the magnetic field treatment, the cells were cultured for another 24 h because apoptosis is a relatively slow process. The cells were stained with the LIVE/DEAD™ Viability/Cytotoxicity Kit and observed on an Olympus fluorescence microscope equipped with a true-color CCD, Qcolor 5.

The MNP dose dependence was also quantified using the standard MTS assay following the manufacturer's instructions. Cells were seeded in 96-well plates (5,000 cells per well, 6 repeats), and incubated with media containing MNPs of various concentrations (0, 5, 10, 50, 100, 500 μg mL⁻¹ based on Fe) for 3 h. After replacing the media with fresh media, cells were exposed to magnets for 1.5 h. 24 h post exposure to the magnetic fields, the MTS solution was added to each well. Cell viability was measured on a microplate reader (Tecan, absorbance at 490 nm).

Imaging endosome/lysosome rupture after MELT treatment. Hela cells were seeded in 24-well glass-bottom plates (20,000 cells per well). After overnight culturing, cells were incubated with Cy3-labeled MNP (0.5 mg mL⁻¹ based on Fe) for 3 h. The cell media were replaced before exposure to the magnetic fields (1 magnet or 2 magnets) for 1.5 h, respectively. The treated cells were immediately stained with Hoechst 33258 and Lysotracker Green. The intracellular pH change was monitored using the pHrodo™ Green AM Intracellular pH Indicator according to manufacturer's instructions. The cells were also stained with Hoechst 33258 and Lysotracker DeepRed for microscopy.

MELT for tumor organoids. In vitro 3D tumor organoids were established by seeding LNCaP cells in the ultra-low attachment 96-well round-bottom plates (ThermoFisher) (10,000 cells per well) and culturing for 4 days. For MNP and MNP-DUPA uptake study, cell organoids were treated with Cy3-labeled MNP or MNP-DUPA (0.5 mg mL⁻¹ Fe) for 24 h. The organoid medium was replaced with fresh medium and imaged on a fluorescence microscope. Fluorescence intensities of tumor organoids were quantified using ImageJ. To test growth inhibition induced by MELT, the organoids were divided into 6 groups: untreated, treated with MF alone, MNP alone, MNP-DUPA alone, MNP+MF, and MNP-DUPA+MF (n=8). The organoids were treated with different MNP agents on day 0 and day 4 for 24 h followed by 1.5 h exposure to magnet fields on day 1 and day 5. The sizes of tumor organoids were measured every day. After 7 days, the cell viability of the tumor organoids was analyzed with the MTS assay.

While illustrative embodiments have been illustrated and described, it will be appreciated that various changes can be made therein without departing from the spirit and scope of the invention.

1. A method for inducing cell death, comprising:
(a) contacting a cell with a magnetic nanoparticle;
(b) after a pre-determined period of time, subjecting the cell to a parallel magnetic field for a time sufficient to achieve cell death.

2. A method for inducing cell death in a subject, comprising:
(a) administering an effective amount of magnetic nanoparticles to a subject;
(b) after a pre-determined period of time, subjecting the subject to a localized parallel magnetic field for a time sufficient to achieve cell death.

3. A method for killing tumor cells in a subject, comprising;
(a) administering an effective amount of magnetic nanoparticles to a subject;
(b) after a pre-determined period of time, subjecting the solid tumor to a localized parallel magnetic field for a time sufficient to achieve cell death and decrease the size of the tumor.

4. The method of claim 1, wherein the magnetic nanoparticle is an iron oxide nanoparticle.

5. The method of claim 1, wherein the magnetic nanoparticle is a dextran- or polyethylene glycol-coated magnetic nanoparticle.

6. The method of claim 1, wherein the magnetic nanoparticle further comprises a targeting agent for selective cell targeting.

7. The method of claim 1, wherein the magnetic nanoparticle is a magnetic resonance imaging agent.

8. (canceled)

9. The method of claim 1, wherein the magnetic nanoparticle is an aqueous colloid of superparamagnetic iron oxide coated with polyglucose sorbitol carboxymethylether.

10. The method of claim 1, wherein the magnetic nanoparticles are administered by local injection, subcutaneous injection, and intravenous injection.

11. The method of claim 3, wherein the magnetic nanoparticles are administered by injection into the solid tumor.

12. The method of claim 1, wherein the parallel magnetic field is provided by a first and a second permanent magnet.

13. The method of claim 1, wherein the parallel magnetic field has a field strength up to about 3 T.

14. The method of claim 1, wherein the cells to be treated are positioned within parallel magnetic field provided by a first and a second permanent magnet.

15. The method of claim 3, wherein the parallel magnetic field provided by a first and a second permanent magnet is positioned on opposite sides of the solid tumor.

16. The method of claim 1, the subject's liver is shielded from the magnetic field.

17. The method of claim 1, wherein the cells are cancer cells.

18. The method of claim 1, wherein the cells are non-cancer cells.

19. The method of claim 3, wherein the solid tumor is a cancerous solid tumor.

20. The method of claim 1, wherein the magnetic particles are endocytosed into the cell.

21. The method of claim 1, wherein the parallel magnetic field induces intracellular self assembly of the magnetic nanoparticles resulting in cell death.

22. (canceled)
